(19) United States
(12) Patent Application Publication
(10) Pub. No.: US 2013/0116291 A1
(43) Pub. Date: May 9, 2013

(54) ASSOCIATION OF XANTHINE OXIDASE INHIBITORS AND STATINS AND USE THEREOF

(71) Applicants: Francesco Melani, Fiesole (IT); Sandro Giuliani, Bagno A Ripoli (IT); Carlo Alberto Maggi, Firenze (IT)

(72) Inventors: Francesco Melani, Fiesole (IT); Sandro Giuliani, Bagno A Ripoli (IT); Carlo Alberto Maggi, Firenze (IT)

(73) Assignee: Menarini International Operations Luxembourg S.A., Lexembourg (DE)

(21) Appl. No.: 13/697,062

(22) PCT Filed: May 6, 2011
(86) PCT No.: PCT/EP2011/057343

§ 371 Date: Jan. 17, 2013

(30) Foreign Application Priority Data
May 10, 2010 (IT) RM 2010 A 000231

(51) Int. Cl. A61K 31/426

(52) U.S. Cl. 514/365

(57) ABSTRACT

The present invention relates to the association of active principles, i.e. of a xanthine oxidase inhibitor with one or more HMG CoA reductase inhibitors, pharmaceutical compositions comprising said active principles, for use in a human or veterinary therapeutic treatment, and methods for the preparation thereof.

### TECHNICAL FIELD OF THE INVENTION

The present invention relates to the association of active principles, i.e. of a xanthine oxidase inhibitor with one or more HMG CoA reductase inhibitors, pharmaceutical compositions comprising said active principles, for use in a human or veterinary therapeutic treatment, and methods for the preparation thereof.

Such associations and compositions proved particularly effective in the treatment of hypercholesterolemia, alone or in association with hyperuricemia or to other disorders in the clinical context of the metabolic syndrome.

### STATE OF THE PRIOR ART

Gout is an invalidating chronic disease characterized by hyperuricemia and deposition of monosodium urate crystals in various tissues, mainly at the joint level and in the kidney. Hyperuricemia e gout are frequently associated to other cardiovascular risk factors such as hypertension hypercholesterolemia and other elements that are part of the metabolic syndrome, like obesity, fasting hyperglycemia, low HDL levels and high triglycerid levels.

Hence, the need to always have novel means of treatment in order to better manage chronic therapy of gout and pathologies frequently correlated thereto.

A xanthine oxidase inhibitor well-known in the literature is allopurinol. More recently, other xanthine oxidase inhibitors have appeared on the market; among them, febuxostat is of particular relevance.

Febuxostat is a powerful non-purine selective inhibitor of xanthine oxidase which in clinical studies has been shown to reduce hyperuricemia more effectively than allopurinol.

Febuxostat is a thiazole derivative having formula (I), belonging to the class of xanthine oxidase inhibitors, and was originally described in EP513379.

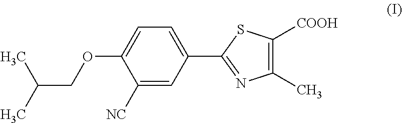 [SMILES: Cc1nc(-c2ccc(OCC(C)C)c(C#N)c2)sc1C(=O)O]

In EP1020454 it is also described a polymorphic form of febuxostat and a process for obtaining it.

In addition to its use as anti-hyperuricemic agent and in the treatment of gout, references are also found to the potential use of febuxostat in other pathologies.

In WO2004060489 it is described the use of xanthine oxidase inhibitors for increasing cardiac contractility in CHF (Chronic Heart Failure) patients.

In WO2007062028 febuxostat is used to reduce the QT interval in patients in which such interval is prolonged, and in the pathologies associated thereto.

In WO2008064015 the use of xanthine oxidase, among which febuxostat, is indicated to preserve renal function.

In WO2007019153 it is claimed the use of some xanthine oxidase inhibitors, among which febuxostat, preferably for the treatment of prehypertension characterized bysystolic pressure between 120 and 139 mmHg and diastolic pressure between 80 and 89 mmHg; here, xanthine oxidase inhibitors seem to be indicated also in the treatment of more marked hypertensions, though results obtained do not seem to be equal to those of already known anti-hypertensive agents.

Hypercholesterolemia is successfully treated with several drugs belonging to different therapeutic classes. Among them, the class of HMG CoA reductase inhibitors must be considered of particular relevance; compounds known as ‘statins’, commonly used in clinical practice and represented by the compounds selected from the group of: atorvastatin, cerivastatin, fluvastatin, lovastatin, pitavastatin, pravastatin, rosuvastatin, simvastatin.

Statins are drugs inhibiting the synthesis of endogenous cholesterol by acting on enzyme 3 hydroxy-3-methyl-glutaryl-CoA reductase, an enzyme converting the 3-hydroxy-3-methyl-glutaryl-coenzime A molecule into mevalonic acid, a cholesterol precursor.

The positive pleiotropic effects of statins on the metabolic syndrome are well-known in the literature.

Atorvastatin was originally described in EP247633; then, in EP409281 and EP1061073 also some specific salts of atorvastatin are reported, among which the calcium or hemicalcium salt thereof are reported.

Lee, S. J. et al in Current Rheumatology Reports (2006), 8(3), 224-230 have also reported the uricosuric effects exhibited by atorvastatin.

### SUMMARY OF THE INVENTION

The present invention is based on the surprising discovery made by the Inventors that the association of a xanthine oxidase inhibitor, in particular febuxostat, or pharmaceutically acceptable salts thereof or polymorphic forms thereof, in combination with one or more HMG CoA reductase inhibitors belonging to the class of statins or pharmaceutically acceptable salts thereof exhibits a synergistic therapeutic effect in the treatment of hypercholesterolemia. In fact, experimental data reported in the present description demonstrate that the therapeutic effect resulting from the association of the two active principles is greater than the sum of the therapeutic effects resulting from the same dosages of each active principle administered alone.

A first object of the present invention is an association of the active principles:

a) the xanthine oxidase inhibitor, febuxostat, or pharmaceutically acceptable salts thereof or polymorphic forms thereof; and

b) one or more HMG CoA reductase inhibitors belonging to the class of statins or pharmaceutically acceptable salts thereof

for use in a human or veterinary therapeutic treatment.

A second object of the present invention is a pharmaceutical composition comprising, as active principle, a mixture of:

a) xanthine oxidase inhibitor febuxostat or pharmaceutically acceptable salts thereof or polymorphic forms thereof; and

b) one or more HMG CoA reductase inhibitors belonging to the class of statins or pharmaceutically acceptable salts thereof

one or more pharmaceutically acceptable excipients and/or carriers and/or diluents, for use in a human or veterinary therapeutic treatment.

Another object of the present invention is a method for the preparation of the composition according to the present description, wherein the active mixture comprising

a) xanthine oxidase inhibitor febuxostat, or pharmaceutically acceptable salts thereof or polymorphic forms thereof; and

b) one or more HMG CoA reductase inhibitors belonging to the class of statins or pharmaceutically acceptable salts thereof is formulated in suitable dosage units with one or more pharmaceutically acceptable excipients.

With respect to the state of the prior art, the present invention entails the advantage of a greater activity in the treatment of hypercholesterolemia compared to that observed using the sole statin or the sole xanthine oxidase inhibitor. Moreover, a further advantage is given by the possibility of obtaining significant effects in the treatment of hypercholesterolemia with a reduced amount of statins with respect to the monotherapy treatment.

### DETAILED DESCRIPTION OF THE INVENTION

The present invention relates to an association of the active principles:

a) xanthine oxidase inhibitor, febuxostat, or pharmaceutically acceptable salts thereof or polymorphic forms thereof; and

b) one or more HMG CoA reductase inhibitors belonging to the class of statins or pharmaceutically acceptable salts thereof

for use in a human or veterinary therapeutic treatment.

By “association” in the present description it is meant an association of the active principles, both in the form of a physical mixture constituted by said active principles in a single dosage unit, and in the form of dosage units physically separated for each active principle, but intended for a concomitant administration. In both cases, association must ensure a synergy of the therapeutic effects obtained from the individual active principles with respect to the effect obtained in monotherapy.

According to the present invention the non-purine xanthine oxidase inhibitor of said association is preferably febuxostat, a thiazole derivative having formula (I), or pharmaceutically acceptable salts thereof or polymorphic forms thereof. Pharmaceutically acceptable salts of xanthine oxidase inhibitors, and in particular of febuxostat, include but are not limited to cations of alkali metals and of alkaline earth metals, such as lithium, sodium, potassium, calcium, magnesium or aluminium salts, or non-toxic derivatives with quaternary ammonium and cations of amines such as ammonium, tetramethylammonium, tetraethylammonium, methylammonium, dimethylammonium, trimethylammonium, or derive from the addition of organic amines such as ethylendiamine, ethanolamine, diethanolamine, piperazine, tromethamine, lysine, arginine and the like.

Polymorphic forms of febuxostat include, but are not limited to the forms described in European Patent EP1020454.

Febuxostat, its salts or polymorphic forms thereof could be obtained or prepared according to methods described in the known art, like e.g. in EP513379.

Polymorphic forms of febuxostat include, but are not limited to the forms described in European Patent EP1020454.

The HMG CoA reductase inhibitors according to the present description belong to the class of statins.

According to an embodiment of the present description one or more of the HMG CoA reductase inhibitors are selected from the group comprising: atorvastatin, cerivastatin, fluvastatin, levostatin, pitavastatin, pravastatin, rosuvastatin, simvastatin or pharmaceutically acceptable salts thereof.

To the ends of the present invention, the HMG CoA reductase inhibitors may be chiral or non-chiral. In case of chiral molecules a single enantiomer, a mixture of enantiomers or diastereoisomers or the racemic mixture could be used. According to the present description those specific stereoisomers, as well as polymorphic forms, which exhibit a greater biological activity are to be preferred.

Pharmaceutically acceptable salts of statins having an acid function in the molecule include but are not limited to cations of alkali metals and of alkaline earth metals, such as lithium, sodium, potassium, calcium, magnesium or aluminium salts, or non-toxic derivatives with quaternary ammonium and cations of amines such as ammonium, tetramethylammonium, tetraethylammonium, methylammonium, dimethylammonium, trimethylammonium, or derive from the addition of organic amines such as ethylendiamine, ethanolamine, diethanolamine, piperazine, tromethamine, lysine, arginine and the like; in the case of atorvastatin, calcium salt is particularly preferred.

In a preferred embodiment the pharmaceutically acceptable salt is atorvastatin calcium.

According to the present description the xanthine oxidase inhibitor, febuxostat, or pharmaceutically acceptable salts thereof or polymorphic forms thereof are associated with one or more HMG CoA reductase inhibitors or pharmaceutically acceptable salts thereof in a weight ratio of febuxostat/HMG CoA reductase inhibitors comprised between 0.1 and 200, or between 0.6 and 10.

E.g., the following amounts, expressed in grams per single dose, could be associated: febuxostat in an amount comprised between 10-200 mg, or better comprised between 25-100 mg, in association with an amount of HMG CoA reductase inhibitors comprised between 1-100 mg, e.g. comprised between 10-40 mg.

Where the association envisages a physical mixture of two compounds, as active principles, having the one an acid function and the other one a basic function, also the forming of an internal salt between the two is possible, in proportion to the respective amounts present in the mixture.

A further embodiment of the present invention relates to pharmaceutical compositions comprising, as active principle, a mixture of:

a) xanthine oxidase inhibitor, febuxostat, or pharmaceutically acceptable salts thereof or polymorphic forms thereof; and

b) one or more HMG CoA reductase inhibitors belonging to the class of statins or pharmaceutically acceptable salts thereof

one or more usual pharmaceutically acceptable excipients and/or additives and/or diluents, for use in a human or veterinary therapeutic treatment.

The HMG CoA reductase inhibitor or the HMG CoA reductase inhibitors to be used according to the above-described composition are selected from the group comprising: atorvastatin, cerivastatin, fluvastatin, levostatin, pitavastatin, pravastatin, rosuvastatin, simvastatin or pharmaceutically acceptable salts thereof.

The pharmaceutical compositions according to the present invention may be formulated in various forms depending on the selected administration route. According to a specific embodiment of the invention, the pharmaceutical composition will be suitable for oral administration of solid forms and may include formulations such as capsules, tablets, pills, powders and granules. In these solid forms the two active principles, the xanthine oxidase inhibitor and the anti-hypercholesterolemic agent (a HMG CoA reductase inhibitor), may be mixed with one or more pharmaceutically acceptable inert excipients. Such excipients may be selected among those commonly known in the state of the art and include, but are not limited to: a) carriers, such as sodium citrate and calcium phosphate, b) fillers, such as starch, lactose, microcrystalline cellulose, sucrose, glucose, mannitol and colloidal silica, c) moistening agents, such as glycerol, d) disintegrating agents, such as alginates, calcium carbonate, starches, derivatives of starch, of cellulose and polyvinylpyrrolidone, silicates and sodium carbonate, e) binders, such as carboxymethyl cellulose, alginates, gelatin, polyvinylpyrrolidone, sucrose, polymeric derivatives of cellulose, starch derivatives, f) retarding agents, such as paraffin, cellulose polymers, fatty acid esters, g) absorption accelerators, such as quaternary ammonium compounds, h) wetting agents and surfactants, such as cetyl alcohol and glycerol monostearate, i) adsorbents, such as bentonite clays and kaolin, k) lubricants, such as talc, calcium stearate, magnesium stearate, polyethylene glycol, sodium lauryl sulfate, sodium stearyl fumarate, j) glidants, such as talc, colloidal silica.

In case the selected compositions constitute the filling of gelatin capsules, the excipients include but are not limited to compounds of the type: lactose, high molecular weight polyethylene glycol, and the like.

Solid-dosage forms may be coated with enteric, gastric coatings, or coatings of other type well-known in the state of the art. They may contain matting agents and may be of the type such as to allow the release of active ingredients only or preferably in a certain section of the intestine, optionally in a delayed manner. Substances capable of allowing such a delayed use include, but are not limited to polymers and waxes.

Liquid forms suitable for oral administration are emulsions, solutions, prepared or extemporary suspensions, syrups and elixirs. Excipients suitable for the formulations according to the present invention in liquid forms for oral use include, but are not limited to diluents commonly used in the art, such as water or other solvents, solubilizing and emulsifying agents selected from ethyl alcohol, polyalcohols, propylene glycol, glycerol, polyethylene glycol and sorbitan esters. These formulations can also contain sweeteners and aromas selected from those well-known in the state of the art.

Compositions suitable for pharmaceutically acceptable parenteral injections may comprise sterile aqueous solutions, sterile dispersions, suspensions or emulsions or powders for a reconstitution in injectable solutions or dispersions; examples of excipients suitable therefor include, but are not limited to aqueous or non-aqueous carriers, diluents, solvents or vehicles selected from: water, ethanol, polyoils (propylene or polyethylene glycol, glycerol, and the like), polyalcohols, isopropyl alcohol, ethyl acetate, benzyl alcohol, benzyl benzoate, propylene glycol, 1.3-butylene glycol, dimethylformamide, vegetable oils (in particular of olive, cotton, peanut, corn, wheat germ, olive, castor, sesame), organic esters such as ethyl oleate or the like.

These compositions may also contain preservatives of antibacterial or antifungal type, selected, yet not exclusively, from: paraben, chlorbutanol, phenol, sorbic acid and the like. It may also be useful to include an isotonic agent, e.g., a sugar, sodium chloride or the like. Moreover, pharmaceutical forms with a delayed absorption may be obtained with agents such as, for instance, yet not exclusively, aluminium monostearate and gelatin.

The suspensions, beside the active principles (xanthine oxidase inhibitors and HMG CoA reductase inhibitors), may contain suspending agents such as, for instance, yet not exclusively, ethoxylated isostearic alcohols, polyethylene sorbitol and sorbitan esters, microcrystalline cellulose, aluminium hydroxide, bentonite, alginates and cellulose derivatives in general or the like.

The right fluidity can be maintained with a coating material such as lecithin, with the maintaining of the right particle sizes in the dispersions or with the use of surfactants.

Also slow-release formulations can be prepared, by the techniques and products well-known in the state of the art.

The associations and compositions according to the invention are extremely effective in the treatment, prophylactic as well as therapeutic, of hypercholesterolemia, in humans or animals.

Hypercholesterolemia can be associated or not associated to other pathologies or syndromes and symptoms. In particular, the association described herein is useful also in the therapeutic treatment of hypercholesterolemia associated to hyperuricemia and/or hyperglycemia.

Symptoms such as hypercholesterolemia, hyperuricemia or hyperglycemia can also be associated, individually or in combination, to specific syndromes like the metabolic syndrome.

By “metabolic syndrome” it is meant a clinical condition accompanied by manifestations such as obesity.

The association described herein can therefore be used in the therapeutic treatment of hypercholesterolemia associated to hyperuricemia and/or hyperglycemia or to other disorders in the context of the metabolic syndrome.

Dosage may vary depending on the patient's age and general conditions, the nature and seriousness of the pathology or disorder and of the administration route and type. Dosage should therefore take into account the specific condition to be treated (e.g., hypercholesterolemia alone or in association with hyperuricemia and/or glycemia), the severity of the condition to be treated, the age, weight and general physical conditions of the specific patient, as well as other drugs that the patient is taking, as is well-known to those skilled in the art. Moreover, it is evident that said effective amount may, when required, be lowered or raised according to the responses of the treated patient and/or according to the assessment of the physician prescribing the compounds of the present invention.

Typically, compositions for oral use in solid form can contain an amount of xanthine oxidase inhibitor, specifically febuxostat, of between 10 and 200 mg per single dose, and preferably of between 25 and 100 mg, and an amount of statin, preferably atorvastatin, and even more preferably atorvastatin calcium salt, of between 1 and 100 mg per single dose, preferably of between 10 and 40 mg.

By the term “dosage unit” in the present description it is meant the unitary formulation for a single administration, e.g. a tablet, capsule, etc.

By “unit dosage” it is meant the amount of active principle for a single administration.

The pharmaceutical mixtures and compositions of the invention could be prepared according to techniques known in the field, both using the previously prepared association of active principles and mixing the individual compounds directly during the preparation of the composition.

In particular, the association of active principles may be obtained by a step of mixing the xanthine oxidase inhibitor, febuxostat, or pharmaceutically acceptable salts thereof or polymorphic forms thereof with one or more HMG CoA reductase inhibitors belonging to the class of statins or pharmaceutically acceptable salts thereof, in a weight ratio comprised between 0.1 and 200, or between 0.6 and 10.

For the preparation of the pharmaceutical compositions described herein the mixture of active principles is formulated in suitable dosage units with one or more pharmaceutically acceptable excipients and additives.

Testing

Testing demonstrating activity of the associations according to the invention is reported hereinafter.

1—Biological Activity Measurement

Hypocholesterolemic activity of atorvastatin, alone or in association with febuxostat, was assessed in Wistar rats having a weight of 125-150 g (Harlan Laboratories, Udine, Italy) subjected to a cholesterol-rich (2%) diet for 6 weeks. After 2 weeks, with increased cholesterolemia values, oral treatment with febuxostat and/or atorvastatin was started; the treatment was carried on for other 4 weeks. Plasma levels of total cholesterol were measured with a standard colorimetric enzymatic method.

Febuxostat and/or atorvastatin were administered orally, by gavage, once per day for 4 weeks, at the doses of 1-2.5-5 mg/kg for febuxostat and of 2.5-5-10mg/kg for atorvastatin, as atorvastatin calcium salt. Plasma levels of total cholesterol were determined at one-week intervals. Blood was collected from a tail vein. In rats fed the cholesterol-rich diet, cholesterolemia more than doubled with respect to control rats, with an increase of about 120% (n=5).

Febuxostat did not significantly modify the cholesterol levels in the 4 weeks of observation, not even at the highest dose thereof (5 mg/kg per day, per os), whereas atorvastatin showed a significant and dose-dependent reduction of the cholesterolemia values already starting from the first week of treatment at the doses of 5 and 10 mg/kg, at 2 weeks reaching an activity peak which remained constant in the successive weeks, with a maximum reduction of 80% at the highest dose. The lowest dose of atorvastatin (2.5 mg/kg as calcium salt, per os daily) reduced only of 25% the total cholesterolemia levels starting from the second week of testing.

The combined administration of febuxostat and atorvastatin yielded maximal results in reducing the high levels of cholesterolemia at the daily doses of 5 mg/kg per os of febuxostat and of 2.5 mg/kg per os of atorvastatin calcium salts, demonstrating a surprising synergistic effect and suggesting that the two compounds have a favourable and unexpected interaction that can yield positive effects in hypercholesterolemia reducing the effective doses of atorvastatin. The results are reported in the following table. These results demonstrate the possible advantages attainable with associations of febuxostat and atorvastatin at therapeutic level in hypercholesterolemia.

| | Diminution of plasma |
|---|---|
| | cholesterol levels in mice |
| Active principle dosage | subjected to treatment |
| Febuxostat 1 mg/Kg | No diminution |
| Febuxostat 2.5 mg/Kg | No diminution |
| Febuxostat 5 mg/Kg | No diminution |
| Atorvastatin 2.5 mg/Kg | 25% |
| Atorvastatin 5 mg/Kg | 80% |
| Atorvastatin 10 mg/Kg | 80% |
| Atorvastatin 2.5 mg/Kg + Febuxostat 5 | 80% |
| mg/Kg | |

### Example 1

tablet for oral administration, containing:

| | | |
|---|---|---|
| febuxostat | 120 | mg |
| atorvastatin calcium | 40 | mg |
| pregelatinized starch (disintegrating binder) | 70 | mg |
| silicified microcrystalline cellulose (filler) | 32.656 | mg |
| croscarmellose sodium (disintegrant) | 10 | mg |
| magnesium stearate (lubricant) | 0.8 | mg |

### Example 2

tablet for oral administration, containing:

| | | |
|---|---|---|
| febuxostat | 80 | mg |
| atorvastatin calcium | 20 | mg |
| pregelatinized starch (disintegrating binder) | 35 | mg |
| silicified microcrystalline cellulose (filler) | 72.256 | mg |
| croscarmellose sodium (disintegrant) | 5 | mg |
| magnesium stearate (lubricant) | 0.4 | mg |

### Example 3

tablet for oral administration, containing:

| | | |
|---|---|---|
| febuxostat | 40 | mg |
| atorvastatin calcium | 10 | mg |
| pregelatinized starch (disintegrating binder) | 35 | mg |
| silicified microcrystalline cellulose (filler) | 85.312 | mg |
| croscarmellose sodium (disintegrant) | 5 | mg |
| magnesium stearate (lubricant) | 0.4 | mg |

The above experimental results and the specific embodiments of the invention made for the testing have the purpose of illustrating the invention, of course without limiting its embodiment to what is reported below.

1. A method of treatment comprising administering active principles:
a) xanthine oxidase inhibitor, febuxostat, or a pharmaceutically acceptable salt thereof or polymorphic form thereof; and
b) one or more HMG CoA reductase inhibitors that are statins or pharmaceutically acceptable salts thereof to a human or veterinary subject.

2. The method of treatment according to claim 1, wherein the active principle (a) is associated with the active principle (b) in a weight ratio of (a)/(b) comprised between 0.1 and 200.

3. The method of treatment according to claim 2, wherein said weight ratio is comprised between 0.6 and 10.

4. The method of treatment association according to claim 1, wherein said HMG CoA reductase inhibitor is selected from the group consisting of: atorvastatin, cerivastatin, fluvastatin, levostatin, pitavastatin, pravastatin, rosuvastatin, simvastatin, and pharmaceutically acceptable salts thereof.

5. The method of treatment according to claim 1, wherein said HMG CoA reductase inhibitor is atorvastatin calcium.

6. The method of treatment according to claim 1, for use in therapeutic treatment of hypercholesterolemia.

7. The method of treatment according to claim 1, for use in therapeutic treatment of hypercholesterolemia associated with hyperuricemia and/or hyperglycemia.

8. The method of treatment according to claim 1, for use in therapeutic treatment of hypercholesterolemia associated with hyperuricemia and/or hyperglycemia in the context of metabolic syndrome.

9. The method of treatment according to claim 1, for use in therapeutic treatment of hypercholesterolemia and associated with disorders of metabolic syndrome.

10. A pharmaceutical composition for use in a human or veterinary therapeutic treatment, the pharmaceutical composition comprising an active mixture of:
a) xanthine oxidase inhibitor, febuxostat, or a pharmaceutically acceptable salt thereof or polymorphic form thereof; and
b) one or more HMG CoA reductase inhibitors that are statins or pharmaceutically acceptable salts thereof
and one or more pharmaceutically acceptable excipients and/or additives and/or diluents.

11. The pharmaceutical composition according to claim 10, wherein said HMG CoA reductase inhibitor is selected from the group consisting of: atorvastatin, cerivastatin, fluvastatin, levostatin, pitavastatin, pravastatin, rosuvastatin, simvastatin, and pharmaceutically acceptable salts thereof.

12. The pharmaceutical composition according to claim 10, wherein said HMG CoA reductase inhibitor is atorvastatin calcium.

13. The pharmaceutical composition according to claim 10, for use in therapeutic treatment of hypercholesterolemia.

14. The pharmaceutical composition according to claim 10, for use in therapeutic treatment of hypercholesterolemia associated with hyperuricemia and/or hyperglycemia.

15. The pharmaceutical composition according to claim 10, for use in therapeutic treatment of hypercholesterolemia associated with hyperuricemia and/or hyperglycemia in the context of metabolic syndrome.

16. The pharmaceutical composition according to claim 10, for use in therapeutic treatment of hypercholesterolemia associated with other disorders of metabolic syndrome.

17. The pharmaceutical composition according to claim 10, wherein said xanthine oxidase inhibitor, febuxostat, is in an amount comprised between 10-200 mg per dosage unit.

18. The pharmaceutical composition according to claim 10, wherein said xanthine oxidase inhibitor, febuxostat, is in an amount comprised between 25-100 mg per dosage unit.

19. The pharmaceutical composition according to claim 10, wherein said HMG CoA reductase inhibitor is in an amount comprised between 1.0-100 mg per dosage unit.

20. The pharmaceutical composition according to claim 10, wherein said HMG CoA reductase inhibitor is in an amount comprised between 10-40 mg per dosage unit.

21. A method for preparation of the pharmaceutical composition according to claim 10, wherein the active mixture
is formulated in suitable dosage units with one or more pharmaceutically acceptable excipients and/or additives and/or diluents.

22. The method of treatment according to claim 5, for use in therapeutic treatment of hypercholesterolemia.

23. The method of treatment according to claim 5, for use in therapeutic treatment of hypercholesterolemia associated with hyperuricemia and/or hyperglycemia.

24. The method of treatment according to claim 5, for use in therapeutic treatment of hypercholesterolemia associated with hyperuricemia and/or hyperglycemia in the context of metabolic syndrome.

25. The method of treatment according to claim 5, for use in therapeutic treatment of hypercholesterolemia and associated with disorders of metabolic syndrome.
